(19) United States
(12) Patent Application Publication
(10) Pub. No.: US 2011/0071174 A1
(43) Pub. Date: Mar. 24, 2011

(54) SUBSTITUTED QUINAZOLINES

(75) Inventors: Bernard Golding, Tyne and Wear (GB); Richard Franklin, Hampshire (GB)

(73) Assignee: Shire LLC

(21) Appl. No.: 12/993,064

(22) PCT Filed: May 13, 2009
(86) PCT No.: PCT/GB09/50515

§ 371 Date: Nov. 16, 2010

(30) Foreign Application Priority Data
May 16, 2008 (GB) 0808953.4

(51) Int. Cl. A61K 31/519; C07D 239/00; C07D 471/04; A61K 31/437; A61P 7/02; A61P 35/00

(52) U.S. Cl. 514/267; 544/250; 546/84; 514/292

(57) ABSTRACT

This invention relates to the discovery of substituted analogues of the selective platelet lowering agent anagrelide with reduced potential for cardiovascular side-effects which should lead to improved patient compliance and safety in the treatment of myeloproliferative diseases. More specifically, the present invention relates to certain imidazoquinazoline derivatives which have the general formula shown below wherein the substituents have the meanings defined in claim 1: and which have utility as platelet lowering agents in humans. The compounds of the present invention function by inhibiting megakaryocytopoeisis and hence the formation of blood platelets.

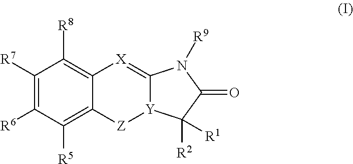

### FIELD OF THE INVENTION

This invention relates to the discovery of substituted analogues of the selective platelet lowering agent anagrelide with reduced potential for cardiovascular side-effects which should lead to improved patient compliance and safety in the treatment of myeloproliferative diseases. More specifically, the present invention relates to certain imidazoquinazoline derivatives which have utility as platelet lowering agents in humans. The compounds of the present invention function by inhibiting megakaryocytopoeisis and hence the formation of blood platelets.

### BACKGROUND OF THE INVENTION

Anagrelide hydrochloride (Agrylin®, Xagrid®) is a novel orally administered imidazoquinazoline which selectively reduces platelet count in humans and is used for such purposes in the treatment of myeloproliferative diseases (MPDs), such as essential thrombocythemia (ET), where an elevated platelet count may put the patient at increased thrombotic risk. The chemical structure of anagrelide, 6,7-dichloro-1,5-dihydroimidazo[2,1-b]-quinazolin-2(3H)-one is shown as the hydrochloride monohydrate in the following formula:

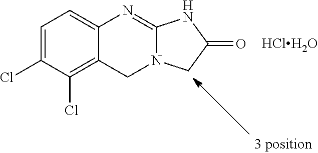

Preparation of anagrelide hydrochloride was referred to in U.S. Pat. Nos. 3,932,407; RE31,617 and 4,146,718.

Anagrelide is a unique, highly selective platelet lowering agent. In vitro studies of human megakaryocytopoiesis suggested that, in vivo, its thrombocytopenic activity results primarily from an inhibitory effect on megakaryocyte maturation. Anagrelide inhibited TPO-induced megakaryocytopoiesis in a dose-dependent manner with an estimated IC₅₀ of ˜26 nM, showing it to be a highly potent agent. Anagrelide does not affect erythroid or myelomonocytic differentiation stimulated by erythropoietin or granulocyte-macrophage colony-stimulating factor, demonstrating the selectivity of this compound against the megakaryocytic lineage.

The drug, which is available in both the U.S. and Europe, has proven to be of considerable clinical value in the treatment of myeloproliferative diseases, such as essential thrombocythemia. Anagrelide was shown to be effective and selective in reducing and maintaining platelet count close to or within the physiological range in patients with thrombocythemia secondary to a myeloproliferative disorder. The time to complete response, defined as a platelet count 600×10⁹/L, ranged from 4 to 12 weeks. In the majority of patients, the platelet count can be reduced and maintained at a dose of 1 to 3 mg/day.

In early volunteer trials, the most frequently reported adverse effects AEs other than headache were palpitations, postural dizziness and nausea. During patient studies, the most frequently reported drug-related AEs were headache, palpitations, oedema/fluid retention, nausea/vomiting, diarrhea, dizziness and abdominal pain. These effects are all likely to arise from the secondary, cardiovascular pharmacology associated with anagrelide resulting from its inhibitory effects on human phosphodiesterase III (PDE III). Anagrelide is a potent PDE III inhibitor with an IC₅₀ value of ˜29 nM (cf. milrinone, a classical PDE III inhibitor, IC₅₀=170-350 nM). Inhibition of myocardial PDE III leads to positive inotropy (increasing of the force of contractions of the heart), increased chronotropy (increase in heart rate), and peripheral vasodilatation. Such cardiovascular manifestations of this inhibition are typically seen with the classical positive inotropes, milrinone and enoximone, and exploited in the short-term acute treatment of congestive heart failure. However, in the treatment of a so-called silent disease (i.e., asymptomatic) such as ET, the cardiovascular side-effects of palpitations and tachycardia associated with anagrelide limit its utility and a significant proportion of patients—reportedly between 25 and 50%—fail to tolerate the drug during long term treatment.

The PDE III inhibitory properties of anagrelide are quite distinct from its platelet lowering anti-megakaryocytic effects. Indeed studies have shown no correlation between potency as a PDE III inhibitor and anti-megakaryocytic effects for anagrelide and its principal pharmacologically active metabolite, 3-hydroxyanagrelide (3-OH anagrelide or 3-HA, formerly known as SPD604 or BCH24426). Surprisingly the latter was found to be over 40-fold more potent than anagrelide as a PDE III inhibitor. With respect to inhibition of megakaryocytopoiesis (and therefore platelet lowering potential) it was however no more potent than the parent drug. Anagrelide's active metabolite, 3-HA, is present in vivo in amounts greatly exceeding those of the parent drug with typical exposures being 2-3 fold greater. Thus by implication 3-OH anagrelide is likely to be a major contributor to the pharmacological actions of the drug.

In addition to the unwanted cardiovascular effects associated with PDE III inhibition, the consequent elevation of cAMP can result in an anti-aggregatory effect. While initially this property may appear to be beneficial in essential thrombocythemia patients predisposed to greater thrombotic risk, such anti-platelet effects, in excess, could have haemorrhagic consequences and on balance may not be desirable. Indeed the haemorrhagic events occasionally seen in ET patients treated with anagrelide might be due to a combination of the anti-aggregatory effects contributed largely by 3-OH anagrelide and an overshooting of platelet reduction, compounded by a synergistic interaction with aspirin that is frequently concomitantly administered. (In some ET patients, plasma concentrations of 3-OH anagrelide have been shown likely to exceed the in vitro IC₅₀ values for inhibition of platelet aggregation by a factor of 3).

The PDE III mediated cardiovascular side-effects associated with anagrelide treatment mean that many patients have to be switched to the only significant alternative therapy, namely that with hydroxyurea. However, this drug is a simple chemical anti-metabolite which inhibits ribonucleoside diphosphate reductase (RNR) with resultant profound effects on DNA synthesis. Ribonucleoside diphosphate reductase catalyzes the conversion of ribonucleosides into deoxyribonucleosides, which are the building blocks of DNA synthesis and repair. Inhibition of ribonucleoside diphosphate reductase explains the cytoreductive and—most importantly—the mutagenic effects of this compound as well as its platelet lowering action. Hydroxyurea is thus officially classified as a “presumed human carcinogen.” As well as possessing the potential to induce leukemic transformation, hydroxyurea is associated with the induction of difficult-to-treat leg ulcers.

Faced with this dilemma in treatment options, there is a clear need for a new agent in the treatment of thrombocythemia which is selective in its effects on megakaryocytopoiesis but with reduced or minimal side effects. While anagrelide offers some selectivity in its mechanism of action, the limitations to its use are those associated with cardiovascular effects resulting from its secondary pharmacology and contributed largely by the active metabolite of anagrelide, 3-hydroxyanagrelide.

The metabolism of anagrelide generally proceeds extremely rapidly, resulting in a less than ideal pharmacokinetic profile of the drug. The typical half-life of anagrelide is just 1.5 hr (2.5 hr for the metabolite) necessitating frequent drug administration (up to 4 times per day). This, combined with the side-effects profile, can lead to poor patient compliance. Furthermore, anagrelide undergoes a large first pass effect (>50%) leading to considerable intersubject variation in achieved exposures and, therefore, potentially variable drug response. Also, exposure to the pharmacologically active metabolite varies dramatically between patients since its formation is dependent on CYP1A, an enzyme whose expression is highly dependent on exposure to inducing agents such as cigarette smoke. Overall, this may result in the need for careful dose titration in patients being treated with anagrelide.

U.S. Pat. No. 4,256,748 discloses a number of imidazo[2,1-b]quinazolin-2(3H)-ones which have an analogous structure to anagrelide and which are said to be effective in the treatment of thromboses resulting from their anti-aggregatory effects on blood platelets mediated by PDE III inhibition. However, this disclosure does not appreciate the entirely separate anti-megakaryocytic potential (reducing platelet numbers) which could be associated with some analogues.

Ideally there is a need for compounds which possess anti-megakaryocytic activity whilst at the same time having a reduced level of PDE III inhibitory activity and therefore unwanted cardiovascular effects.

It is an aim of the present invention to overcome various disadvantages of or to improve on the properties of prior art compounds. Thus it is an aim of the invention to provide an anagrelide derivative which has improved activity and/or reduced cardiovascular toxicity relative to prior art compounds in the treatment of diseases for which modulation of megakaryocytopoeisis provides an efficacious treatment. The compounds of the present invention are especially beneficial because they display less inhibitory activity towards phosphodiesterase III (PDE III) and yet surprisingly still retain their anti-megakarycocytic and hence platelet lowering properties.

It is also desirable that the compounds of the present invention should have an improved pharmacokinetic profile to aid patient compliance and ensure consistency of therapeutic response. It is thus a further aim to provide compounds with a good duration of action i.e. long half-life in vivo. Additionally it is a further aim to provide compounds that are available via relatively convenient synthetic processes.

The compounds described in relation to the present invention satisfy some or all of the above aims.

### SUMMARY OF THE INVENTION

We have found that analogues of anagrelide in which the principal site of metabolism is blocked by an appropriate group are likely not only to have improved pharmacokinetics but also a better side effect profile. This would be expected to lead to better tolerability and improved patient compliance enabling a broader spectrum of patients to be effectively treated.

The compounds of the present invention are surprisingly beneficial for two reasons: they have a dramatically lower PDE III inhibitory activity than 3-hydroxyanagrelide, yet still retain potent anti-megakaryocytic activity. Indeed these compounds have therapeutic indices which are likely to be much more favorable than that for anagrelide itself.

According to one aspect of the present invention, there is provided a compound of Formula (I) or a pharmaceutically acceptable salt or solvate thereof:

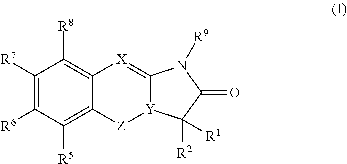

wherein:
X is —C(R¹⁰)=, Y is N and Z is —C(R³)(R⁴)—; or X is —N═ and ZY is C(R³)═C;
R¹, R², R³ and R⁴ independently represent hydrogen or a blocking group which functions to prevent metabolic reaction either directly or indirectly at the carbon atom to which R¹ and R² are attached;
or R¹ and R², and/or R³ and R⁴ together with the carbon to which they are attached form a blocking group which functions to prevent metabolic reaction at the carbon atom to which R¹ and R² are attached, the remainder of groups R¹ to R⁴ being hydrogen;
R⁵, R⁶, R⁷, R⁸ and R¹⁰ are each independently selected from hydrogen, R^{a} and R^{b};
R⁹ is hydrogen, C₁₋₆ alkyl or a Group I or Group II metal ion;
R^{a} is selected from C₁₋₆ alkyl and C₂₋₆ alkenyl, either of which is optionally substituted with 1, 2, 3, 4 or 5 R^{b};
R^{b} is selected from halo, trifluoromethyl, cyano, nitro, —OR^{c}, —C(O)R^{c}, —C(O)OR^{c}, —OC(O)R^{c}, —S(O)₁R^{c}, —N(R^{c})R^{d}, —C(O)N(R^{c})R^{d}, —N(R^{c})C(O)R^{d}, —S(O)₁N(R^{c})R^{d} and —N(R^{c})S(O)₁R^{d};
R^{c} and R^{d} are each independently hydrogen or R^{e};
R^{e} is selected from C₁₋₆ alkyl and C₂₋₆ alkenyl, either of which is optionally substituted with 1, 2, 3, 4 or 5 substituents independently selected from halo, cyano, amino, hydroxy, nitro and C₁₋₆ alkoxy; and
1 is 0, 1 or 2;
and wherein at least one of R¹, R², R³ and, where present, R⁴ is not hydrogen.

The invention therefore provides compounds of the following Formulae:

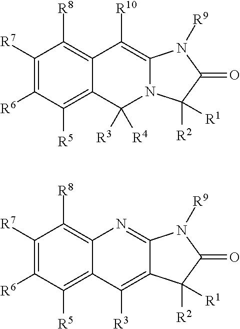

or, in each case, a pharmaceutically acceptable salt or solvate thereof.

In an embodiment:

R¹ and R² are independently selected from the group comprising: H; cyano; C₁₋₆ alkyl, SC₁₋₆ alkyl, C₂₋₆ alkenyl, C₂₋₆ alkynyl, C₃₋₈ cycloalkyl wherein said alkyl, alkenyl, alkynyl or cycloalkyl groups may be optionally substituted by 1 to 5 groups chosen independently from the group comprising: halo, hydroxyl, cyano, nitro, C₁₋₄ alkylsulphonyl and COOH; C₁₋₆ hydroxyalkyl; C₁₋₆ carboxyalkyl; and sulphide;
or R¹ and R² together with the carbon to which they are attached form a C₃₋₈ carbocyclic ring which may be optionally substituted by 1 to 5 groups chosen independently from the group comprising: halo, hydroxyl, cyano, nitro, C₁₋₄ haloalkyl, C₁₋₄ alkylsulphonyl and COOH;
or R¹ and R² together represent a C₂₋₆ alkenyl or C₂₋₆ alkynyl group bound through a double bond to the ring to which it is attached and which may be optionally substituted by one to three groups independently selected from the group comprising: halo, hydroxyl, cyano, C₁₋₄ haloalkyl and COOH.

In a preferred set of compounds, R¹ is an optionally substituted C₁₋₄ alkyl or C₃₋₈ cycloalkyl group.

In a preferred set of compounds, R² is an optionally substituted C₁₋₄ alkyl group or C₃₋₈ cycloalkyl.

In another preferred set of compounds, R¹ and R² together form an optionally substituted C₃₋₈ cycloalkyl group. Most preferably this is a cyclopropyl group.

Other preferred compounds are those in which at least one of R¹ and R² is —C(H)ₙ(F)ₘ or —C(H)ₙ(F)ₘ—C(H)ₚ(F)_{q}, where m=2 or 3, and n=(3-m); and p=2 or 3, and q=(3-p).

More preferably at least one of R¹ and R² is CF₃ or CHF₂. Most preferably, at least one of R¹ and R² is CF₃.

In an embodiment, R¹ is preferably methyl, cyclopropyl, CF₃ or CHF₂. More preferably, R¹ is methyl or cyclopropyl. Most preferably, R¹ is methyl. In an embodiment, R² is preferably methyl, cyclopropyl, CF₃ or CHF₂. More preferably R² is methyl or cyclopropyl. Most preferably R² is methyl.

Another preferred group of compounds is those in which neither R¹ nor R² is hydrogen. Amongst these, it is preferred when R¹ and R² are both independently selected from the group comprising: cyano, C₁₋₆ alkyl, C₂₋₆ alkenyl, C₂₋₆ alkynyl, in which the alkyl, alkenyl, and alkynyl groups may be optionally substituted;

or wherein R¹ and R² together with the carbon to which they are attached form an optionally substituted C₃₋₈ carbocyclic ring
or wherein R¹ and R² together represent an optionally substituted C₂₋₆ alkenyl or C₂₋₆ alkynyl group.

In a particular set of compounds, R¹ and R² are each methyl or together form methylene; or R¹ and R², taken together with the carbon atom to which they are attached, form cyclopropyl.

In compounds in which Z is —C(R³)═, R³ may be, for example, selected from the group comprising: H; cyano; C₁₋₆ alkyl, SC₁₋₆ alkyl, C₂₋₆ alkenyl, C₂₋₆ alkynyl, C₃₋₈ cycloalkyl wherein said alkyl, alkenyl, alkynyl or cycloalkyl groups may be optionally substituted by 1 to 5 groups chosen independently from the group comprising: halo, hydroxyl, cyano, nitro, C₁₋₄ alkylsulphonyl and COOH; C₁₋₆ hydroxyalkyl; C₁₋₆ carboxyalkyl; and sulphide. In an embodiment, R³ is H or C₁₋₆ alkyl. Preferably, R³ is H.

In compounds in which Z is —C(R³)(R⁴)—, R³ and R⁴ may be, for example, as defined below:

R³ and R⁴ are independently selected from the group comprising: H; cyano; C₁₋₆ alkyl, SC₁₋₆ alkyl, C₂₋₆ alkenyl, C₂₋₆ alkynyl, C₃₋₈ cycloalkyl wherein said alkyl, alkenyl, alkynyl or cycloalkyl groups may be optionally substituted by 1 to 5 groups chosen independently from the group comprising: halo, hydroxyl, cyano, nitro, C₁₋₄ alkylsulphonyl and COOH; C₁₋₆ hydroxyalkyl; C₁₋₆ carboxyalkyl; and sulphide;
or R³ and R⁴ together with the carbon to which they are attached form a C₃₋₈ carbocyclic ring which may be optionally substituted by 1 to 5 groups chosen independently from the group comprising: halo, hydroxyl, cyano, nitro, C₁₋₄ haloalkyl, C₁₋₄ alkylsulphonyl and COOH;
or R³ and R⁴ together represent a C₂₋₆ alkenyl or C₂₋₆ alkynyl group bound through a double bond to the ring to which it is attached and which may be optionally substituted by one to three groups independently selected from the group comprising: halo, hydroxyl, cyano, C₁₋₄ haloalkyl and COOH.

In an embodiment, R³ is H or C₁₋₆ alkyl. Preferably, R³ is H.

In an embodiment, R⁴ is H or C₁₋₆ alkyl. Preferably, R⁴ is H.

In a particular embodiment, the compound is selected from compounds of the following Formulae:

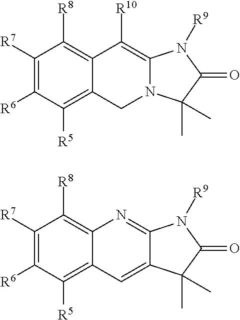

or, in each case, a pharmaceutically acceptable salt or solvate thereof.

In a particular embodiment, the compound is selected from compounds of the following Formulae:

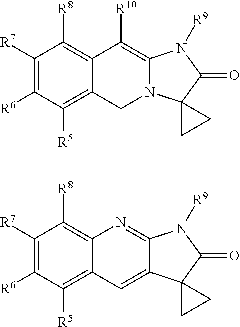

or, in each case, a pharmaceutically acceptable salt or solvate thereof.

In an embodiment R^{a} is C₁₋₆ alkyl optionally substituted with 1, 2, 3, 4 or 5 R^{b}.

In an embodiment R^{a} is C₁, C₂, C₃ or C₄ alkyl, any of which is optionally substituted with 1, 2 or 3 R^{b}.

In an embodiment R^{b} is selected from halo, trifluoromethyl, cyano, nitro, —OR^{c}, —C(O)R^{c}, —C(O)OR^{c}, —OC(O)R^{c}, —S(O)₁R^{c}, —N(R^{c})R^{d}, —C(O)N(R^{c})R^{d}, —N(R^{c})C(O)R^{d}, —S(O)₁N(R^{c})R^{d} and —N(R^{c})S(O)₁R^{d}; wherein R^{c} and R^{d} are each independently hydrogen or C₁₋₆ alkyl optionally substituted with 1, 2, 3, 4 or 5 substituents independently selected from halo, cyano, amino, hydroxy, nitro and C₁₋₆ alkoxy.

In an embodiment R^{b} is selected from fluoro, chloro, bromo, iodo, trifluoromethyl, cyano, nitro, —OR^{c}—C(O)R^{c}, —C(O)OR^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy.

In an embodiment, two of R⁵, R⁶, R⁷ and R⁸ are hydrogen, and the other two are independently selected from R^{a} and R^{b}. Of mention are compounds in which R⁷ and R⁸ are each hydrogen.

In an embodiment, three of R⁵, R⁶, R⁷ and R⁸ are hydrogen, and the other is selected from R^{a} and R^{b}. Of mention are compounds in which R⁷ and R⁸ are each hydrogen.

In an embodiment, R⁵ and R⁶ are each independently selected from fluoro, chloro, bromo and iodo; and R⁷ and R⁸ are each hydrogen.

In an embodiment, R⁵ and R⁶ are each chloro; and R⁷ and R⁸ are each hydrogen.

In an embodiment R⁹ is H. In another embodiment, R⁹ is hydrogen, C₁₋₆ alkyl or a Group I metal ion. In an alternative embodiment, R⁹ is C₁₋₆ alkyl and, in this case, the PDE III inhibiting activity is effectively eliminated. Me represents a particularly preferred alkyl substituent. In another alternative embodiment, R⁹ is a Group I metal ion and, in this case the compounds show significantly improved water solubility. Sodium represents a particularly preferred Group I metal.

In an embodiment, R¹⁰ is hydrogen or C₁₋₄ alkyl optionally substituted with 1, 2 or 3 substituents independently selected from halo, cyano, amino, hydroxy, nitro and C₁₋₄ alkoxy.

In an embodiment, R¹⁰ is hydrogen.

It has also been found that the individual enantiomers of the present compounds show efficacy. The present invention therefore also relates to both the resolved optical isomers of such compounds as well as mixtures of enantiomers. For the purposes of comparison of the compounds of the present invention with anagrelide, the correct comparison is that made with the PDE III inhibitory activity of the 3-hydroxy metabolite of anagrelide since this is the predominant component in plasma after anagrelide treatment.

Regarding the use of the compounds of the invention in humans, there is provided:

a pharmaceutical composition comprising a compound of formula (I), or a pharmaceutically acceptable salt or solvate thereof, together with a pharmaceutically acceptable diluent or carrier, which may be adapted for oral, parenteral or topical administration;
a compound of formula (I), or a pharmaceutically acceptable salt or solvate thereof, or a pharmaceutical composition containing any of the foregoing, for use as a medicament;
the use of a compound of formula (I), or a pharmaceutically acceptable salt or solvate thereof in the manufacture of a medicament for the treatment of a disease selected from: myeloprolific diseases and/or generalised thrombotic diseases; and
a method of treating a disease selected from: myeloproliferative diseases and/or generalised thrombotic diseases in a human, which comprises treating said human with an effective amount of a compound of formula (I), or a pharmaceutically acceptable salt or solvate thereof, or with a pharmaceutical composition containing any of the foregoing.

The present invention also encompasses a method of treating a patient having essential thrombocythemia or high blood platelet count, which method comprises administering to the patient a therapeutically effective amount of a compound of the present invention.

Another embodiment of the present invention includes a method of reducing blood platelet count within a patient, which method comprises administering to the patient a therapeutically effective amount of a compound of the present invention.

The present invention encompasses providing the compounds of the present invention for the methods listed above, among others, wherein cardiotoxicity is reduced compared to using anagrelide.

Separately, we have found that both (R) and (S) compounds show good anti-megakaryocytic activity whilst showing significantly reduced PDE III inhibition relative to 3-OH anagrelide. We thus expect that the compounds will have utility in treating myeloproliferative diseases.

Accordingly, the invention also includes the use of a compound of the invention, or a pharmaceutically acceptable salt or solvate thereof in the manufacture of a medicament for the treatment of myeloprolific diseases.

The invention thus also extends to a method of treating myeloproliferative diseases in a human, which comprises treating said human with an effective amount of a compound of the invention, or a pharmaceutically acceptable salt or solvate thereof, or with a pharmaceutical composition containing any of the foregoing.

The present invention also encompasses pharmaceutical compositions comprising a compound or pharmaceutically acceptable salt of a compound of the present invention and a pharmaceutically acceptable carrier.

### DETAILED DESCRIPTION OF THE INVENTION

The present invention is directed to 3-substituted analogues of the established platelet lowering agent anagrelide. Substitution at the 3-position of the anagrelide molecule would be expected to block or hinder the principal site of metabolism and potentially preclude the formation of the highly potent PDE III inhibitor 3-OH anagrelide while substitution at the 1-position has surprisingly been found to abolish PDE III inhibition. The compounds of the present invention retain the anti-megakaryocytic properties (hence platelet lowering activity) of the parent drug molecule but have reduced PDE III inhibitory properties and hence lower potential for unwanted cardiovascular and anti-aggregatory side-effects. They also have the potential for improved pharmacokinetic characteristics as the result of inhibition of metabolism.

The pharmaceutically acceptable acid addition salts of certain of the compounds of formula (I) may also be prepared in a conventional manner. For example, a solution of the free base is treated with the appropriate acid, either neat or in a suitable solvent, and the resulting salt isolated either by filtration or by evaporation under reduced pressure of the reaction solvent. For a review on suitable salts, see “Handbook of Pharmaceutical Salts: Properties, Selection, and Use” by Stahl and Wermuth (Wiley-VCH, Weinheim, Germany, 2002).

### Definition of Terms: —

Halo means a group selected from: fluoro, chloro, bromo or iodo.

The term “alkyl” as used herein as a group or a part of a group refers to a straight or branched hydrocarbon chain containing the specified number of carbon atoms. For example, C₁₋₁₀ alkyl means a straight or branched alkyl containing at least 1 and at most 10 carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl. A C₁₋₁₄ alkyl group is one embodiment, for example methyl, ethyl, n-propyl, isopropyl, n-butyl, isobutyl or t-butyl.

The term “cycloalkyl” as used herein refers to a non-aromatic monocyclic hydrocarbon ring of 3 to 8 carbon atoms such as, for example, but not limited to, cyclopropyl, cyclobutyl, cyclopentyl, cyclohexyl or cycloheptyl.

The term “spirocyclic” as used herein refers to a ring system joined to a second ring system at one carbon atom.

The term “alkoxy” as used herein refers to a straight or branched hydrocarbon chain group containing oxygen and the specified number of carbon atoms. For example, C₁₋₆ alkoxy means a straight or branched alkoxy containing at least 1 and at most 6 carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy. A C₁₋₄ alkoxy group is one embodiment, for example methoxy, ethoxy, propoxy, prop-2-oxy, butoxy, but-2-oxy or 2-methylprop-2-oxy.

The term “hydroxyalkyl” as used herein as a group refers to a straight or branched hydrocarbon chain containing the specified number of carbon atoms, which is substituted by 1-3 hydroxyl groups. For example, C₁₋₄ hydroxyalkyl means a straight or branched alkyl chain containing from 1 to 4 carbon atoms and at least one hydroxyl group; examples of such group include hydroxymethyl, hydroxyethyl, hydroxypropyl, hydroxyisopropyl, hydroxybutyl and the like.

The term “alkenyl” as used herein as a group or a part of a group refers to a straight or branched hydrocarbon chain containing the specified number of carbon atoms and containing at least one double bond. For example, the term “C₂₋₆ alkenyl” means a straight or branched alkenyl containing at least 2 and at most 6 carbon atoms and containing at least one double bond.

Examples of “alkenyl” as used herein include, but are not limited to, ethenyl, 2-propenyl, 3-butenyl, 2-butenyl, 2-pentenyl, 3-pentenyl, 3-methyl-2-butenyl, 3-methylbut-2-enyl, 3-hexenyl and 1,1-dimethylbut-2-enyl. It will be appreciated that in groups of the form —O—C₂₋₆ alkenyl, the double bond is preferably not adjacent to the oxygen.

The term “alkynyl” as used herein as a group or a part of a group refers to a straight or branched hydrocarbon chain containing the specified number of carbon atoms and containing at least one triple bond. For example, the term “C₂₋₆ alkynyl” means a straight or branched alkynyl containing at least 2 and at most 6 carbon atoms and containing at least one triple bond. Examples of “alkynyl” as used herein include, but are not limited to, ethynyl, 2-propynyl, 3-butynyl, 2-butynyl, 2-pentynyl, 3-pentynyl, 3-methyl-2-butynyl, 3-methylbut-2-ynyl, 3-hexynyl and 1,1-dimethylbut-2-ynyl. It will be appreciated that in groups of the form —O—C₂₋₆ alkynyl, the triple bond is preferably not adjacent to the oxygen. The term “halo” refers to halogens such as fluorine, chlorine, bromine or iodine atoms.

The term “sulfide” refers to a radical of Rₐ—S—R_{b}, wherein a sulfur atom is covalently attached to two hydrocarbon chains, Rₐ and R_{b}, wherein the two hydrocarbon chains may be, for example, but not limited to, any discussed above.

The compounds of the invention, i.e. those of formula (I), possess antimegakaryocytic activity in humans. They may be particularly useful in the treatment of myeloprolific diseases. The compounds may also find utility in the treatment of generalised thrombotic diseases.

It is to be appreciated that references to treatment include prophylaxis as well as the alleviation of established symptoms of a condition. “Treating” or “treatment” of a state, disorder or condition includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

Myeloproliferative diseases which may be treatable with the compounds of the present invention include: essential thrombocythemia, polycythema vera, chronic idiopathic myelofibrosis, chronic myeloid leukaemia with residual thrombocytosis, reactive thrombocytosis immediately preceding a surgical procedures, as an immediate or post operative preventative measures to minimise the risk of thrombus formation during or post surgery.

Thrombotic cardiovascular diseases (TCVD) (i.e. patients at increased generalised thrombotic risk) which may also be treatable with the compounds of the present invention include: myocardial infarct (heart attack) thrombotic stroke, patients having undergone coronary stent placement.

The compounds of the present invention may find utility for the reduction of atherothrombotic events as follows: recent MI, recent stroke or established peripheral arterial disease, acute coronary syndrome (unstable angina/non-Qwave MI), cardiovascular death, MI, stroke, and refractory ischemia.

It is to be understood that compounds of formula (I) may contain one or more asymmetric carbon atoms, thus compounds of the invention can exist as two or more stereoisomers.

Included within the scope of the present invention are all stereoisomers such as enantiomers and diastereomers, all geometric isomers and tautomeric forms of the compounds of formula (I), including compounds exhibiting more than one type of isomerism, and mixtures of one or more thereof.

Unexpectedly it has been found that stable metal salts can be prepared following deprotonation at the 1-position of the quinazoline ring structure. The value of such salts is seen in their relatively much greater aqueous solubility than the corresponding HBr salts. This is likely to facilitate the rapid dissolution and quantitative absorption of these generally poorly water soluble compounds and so represent a major clinical advantage. These salts are Group I metal salts and most usually are sodium or potassium salts.

Geometric isomers may be separated by conventional techniques well known to those skilled in the art, for example, by chromatography and fractional crystallisation.

Stereoisomers may be separated by conventional techniques known to those skilled in the art—see, for example, “Stereochemistry of Organic Compounds” by E L Eliel (Wiley, New York, 1994).

The compounds of formula I can be prepared using literature techniques and in an analogous manner to those described in Formula Scheme I and Formula Scheme II in U.S. Pat. No. 4,256,748.

By way of illustration, and without limitation, a compound of the invention may be obtained according to the following reaction schemes:

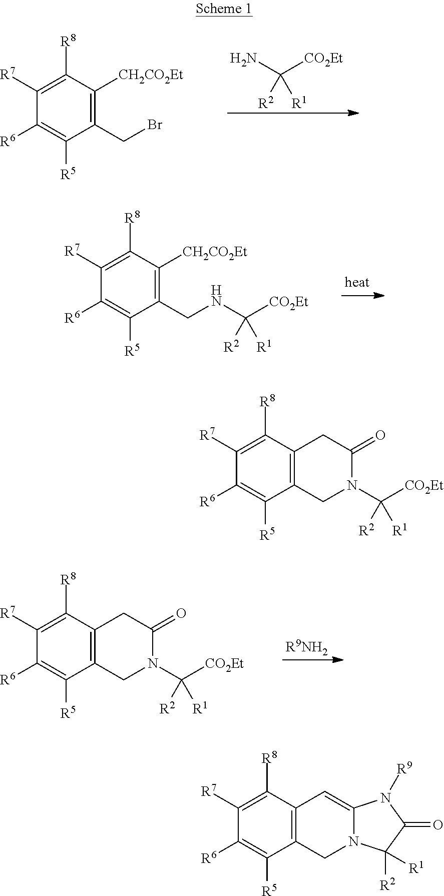

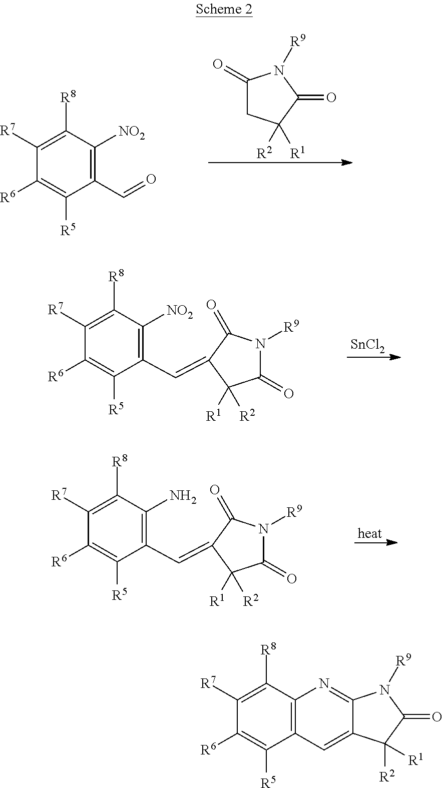

A person skilled in the art will be aware of variations of, and alternatives to, the processes referred to above and to those in U.S. Pat. No. 4,256,748 which allow the individual compounds defined by formula (I) to be obtained.

It will also be appreciated by a person skilled in the art that the compounds of the invention could be made by adaptation of the methods herein described and/or adaptation of methods known in the art, for example the art described herein, or using standard textbooks such as “Comprehensive Organic Transformations—A Guide to Functional Group Transformations”, R C Larock, Wiley-VCH (1999 or later editions), “March's Advanced Organic Chemistry—Reactions, Mechanisms and Structure”, MB Smith, J. March, Wiley, (5th edition or later) “Advanced Organic Chemistry, Part B, Reactions and Synthesis”, F A Carey, R J Sundberg, Kluwer Academic/Plenum Publications, (2001 or later editions), “Organic Synthesis—The Disconnection Approach”, S Warren (Wiley), (1982 or later editions), “Designing Organic Syntheses” S Warren (Wiley) (1983 or later editions), “Guidebook To Organic Synthesis” R K Mackie and D M Smith (Longman) (1982 or later editions), etc., and the references therein as a guide.

It will also be apparent to a person skilled in the art that sensitive functional groups may need to be protected and deprotected during synthesis of a compound of the invention. This may be achieved by conventional methods, for example as described in “Protective Groups in Organic Synthesis” by T W Greene and P G M Wuts, John Wiley & Sons Inc (1999), and references therein.

Compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

They may be administered alone or in combination with one or more other compounds of the invention or in combination with one or more other drugs. Generally, they will be administered as a formulation in association with one or more pharmaceutically acceptable excipients.

Pharmaceutically acceptable excipients include one or more of: anti-oxidants, colourants, flavouring agents, preservatives and taste-masking agents.

Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995). The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

The methods by which the compounds may be administered include oral administration by capsule, bolus, tablet, powders, lozenges, chews, multi and nanoparticulates, gels, solid solution, films, sprays, or liquid formulation. Liquid forms include suspensions, solutions, and syrups. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid preparation, for example, from a sachet.

The compounds may also be administered topically to the skin or mucosa, that is dermally or transdermally. Typical formulations for this purpose include pour-on solutions, sprays, powder formulations, gels, hydrogels, lotions, creams, ointments, films and patches, and implants.

The compounds can also be administered parenterally, or by injection directly into the blood stream, muscle or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Formulations may be immediate and/or modified controlled release. Controlled release formulations include Modified release formulations include: delayed-, sustained-, and pulsed-release.

### Dosages

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

In general however a suitable dose will be in the range of from about 0.001 to about 50 mg/kg of body weight per day, in a further embodiment, of from about 0.001 to about 5 mg/kg of body weight per day; in a further embodiment of from about 0.001 to about 0.5 mg/kg of body weight per day and in yet a further embodiment of from about 0.001 to about 0.1 mg/kg of body weight per day. In further embodiments, the ranges can be of from about 0.1 to about 750 mg/kg of body weight per day, in the range of 0.5 to 60 mg/kg/day, and in the range of 1 to 20 mg/kg/day.

The desired dose may conveniently be presented in a single dose or as divided doses administered at appropriate intervals, for example as one, two, three, four or more doses per day. If the compounds are administered transdermally or in extended release form, the compounds could be dosed once a day or less.

The compound is conveniently administered in unit dosage form; for example containing 0.1 to 50 mg, conveniently 0.1 to 5 mg, most conveniently 0.1 to 5 mg of active ingredient per unit dosage form. In yet a further embodiment, the compound can conveniently administered in unit dosage form; for example containing 10 to 1500 mg, 20 to 1000 mg, or 50 to 700 mg of active ingredient per unit dosage form.

1. A compound of Formula (I) or a pharmaceutically acceptable salt or solvate thereof: 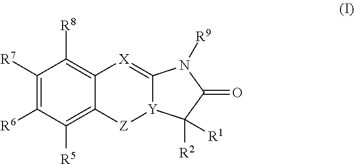
wherein:
X is —C(R¹⁰)═, Y is N and Z is —C(R³)(R⁴)—; or X is —N═ and ZY is C(R³)═C;
R¹, R², R³ and R⁴ independently represent hydrogen or a blocking group which functions to prevent metabolic reaction either directly or indirectly at the carbon atom to which R¹ and R² are attached;
or R¹ and R², and/or R³ and R⁴ together with the carbon to which they are attached form a blocking group which functions to prevent metabolic reaction at the carbon atom to which R¹ and R² are attached, the remainder of groups R¹ to R⁴ being hydrogen;
R⁵, R⁶, R⁷, R⁸ and R¹⁰ are each independently selected from hydrogen, R^{a} and R^{b};
R⁹ is hydrogen, C₁₋₆ alkyl or a Group I or Group II metal ion;
R^{a} is selected from C₁₋₆ alkyl and C₂₋₆ alkenyl, either of which is optionally substituted with 1, 2, 3, 4 or 5 R^{b};
R^{b} is selected from halo, trifluoromethyl, cyano, nitro, —OR^{c}, —C(O)R^{c}, —C(O)OR^{c}, —OC(O)R^{c}, —S(O)₁R^{c}, —N(R^{c})R^{d}, —C(O)N(R^{c})R^{d}, —N(R^{c})C(O)R^{d}, —S(O)₁N(R^{c})R^{d} and —N(R^{c})S(O)₁R^{d};
R^{c} and R^{d} are each independently hydrogen or R^{e};
R^{e} is selected from C₁₋₆ alkyl and C₂₋₆ alkenyl, either of which is optionally substituted with 1, 2, 3, 4 or 5 substituents independently selected from halo, cyano, amino, hydroxy, nitro and C₁₋₆ alkoxy; and
1 is 0, 1 or 2;
and wherein at least one of R¹, R², R³ and, where present, R⁴ is not hydrogen.

2. A compound according to claim 1, wherein X is —C(R¹⁰)═, Y is N and Z is —C(R³)(R⁴)—.

3. A compound according to claim 2, wherein R¹⁰ is hydrogen.

4. A compound according to claim 2, wherein R³ and R⁴ are each hydrogen.

5. A compound according to claim 1, wherein X is —N═ and ZY is C(R³)═C.

6. A compound according to claim 5, wherein R³ is hydrogen.

7. A compound according to claim 1, wherein R¹ and R² are each methyl.

8. A compound according to claim 1, wherein R¹ and R² taken together with the carbon atom to which they are attached, form cyclopropyl.

9. A compound according to claim 1, wherein R⁵ and R⁶ are each independently selected from fluoro, chloro, bromo and iodo.

10. A compound according to claim 1, wherein R⁷ and R⁸ are each hydrogen.

11. A compound according to claim 1, wherein R⁹ is hydrogen, methyl or sodium.

12. A compound according to claim 11, wherein R⁹ is hydrogen.

13. A pharmaceutical composition comprising a compound of claim 1, or a pharmaceutically acceptable salt or solvate thereof, together with a pharmaceutically acceptable diluent or carrier, which may be adapted for oral, parenteral or topical administration.

14. A compound of formula (I) as defined in claim 1, or a pharmaceutically acceptable salt or solvate thereof, or a pharmaceutical composition containing any of the foregoing, for use as a medicament.

15. A compound of formula (I) as defined in claim 1, or a pharmaceutically acceptable salt or solvate thereof, or a pharmaceutical composition containing any of the foregoing, for use in the treatment of a disease selected from: myeloprolific diseases and generalised thrombotic diseases.

16. The use of a compound of formula (I) as defined in claim 1, or a pharmaceutically acceptable salt or solvate thereof in the manufacture of a medicament for the treatment of a disease selected from: myeloprolific diseases and generalised thrombotic diseases.

17. A method of treating a disease selected from: myeloprolific diseases and generalised thrombotic diseases in a human, which comprises treating said human with an effective amount of a compound of formula (I) as defined in claim 1, or a pharmaceutically acceptable salt or solvate thereof, or with a pharmaceutical composition containing any of the foregoing.

18. Use of a compound of formula (I) as defined in claim 1 for the reduction of platelet count.
